(19) United States
(12) Patent Application Publication
(10) Pub. No.: US 2025/0308681 A1
(43) Pub. Date: Oct. 2, 2025

(54) SYSTEM AND METHOD FOR DETECTING UNKNOWN OBJECTS PROXIMATE A PATIENT

(71) Applicant: Welch Allyn, Inc., Skaneateles Falls, NY (US)

(72) Inventors: John A. Lane, Venice, FL (US); Corinn C. Fahrenkrug, Chittenango, NY (US); David E. Quinn, Auburn, NY (US); Tyson B. Whitaker, Arden, NC (US); Gene J. Wolfe, Pittsford, NY (US); WonKyung McSweeney, Manlius, NY (US); Danielle R. Endres, Syracuse, NY (US)

(73) Assignee: Welch Allyn, Inc., Skaneateles Falls, NY (US)

(21) Appl. No.: 19/092,236

(22) Filed: Mar. 27, 2025

Related U.S. Application Data
(60) Provisional application No. 63/570,294, filed on Mar. 27, 2024.

(51) Int. Cl. G16H 40/20; G16H 30/40; G16H 40/63

(52) U.S. Cl. CPC G16H 40/20; G16H 30/40; G16H 40/63

(57) ABSTRACT

A system for detecting an unknown object in a region of interest, the system including: an image sensor; a microphone; and a controller coupled to the image sensor and the microphone for receiving a vocal instruction from a caregiver and for analyzing images from the image sensor in response to the vocal instruction. The controller may be configured to detect and identify the unknown object within the analyzed images. The controller may also monitor the object according to a monitoring protocol. The instruction may identify the region of interest in which the object may be located within the analyzed images. The controller may monitor the region of interest according to a monitoring protocol.

### CROSS REFERENCE TO RELATED APPLICATION

This application claims priority under 35 U.S.C. § 119(e) upon U.S. Provisional Patent Application No. 63/570,294, entitled “SYSTEM AND METHOD FOR DETECTING UNKNOWN OBJECTS PROXIMATE A PATIENT” filed on Mar. 27, 2024, by John A. Lane et al., the entire disclosure of which is incorporated herein by reference.

### FIELD OF THE DISCLOSURE

The present disclosure generally relates to a system and method for detecting and/or identifying unknown objects in a healthcare setting, and more particularly to a system and method for detecting and monitoring objects and/or regions of interest.

### SUMMARY OF THE DISCLOSURE

According to one aspect of the present disclosure, a system is provided for detecting an unknown object in a region of interest, the system including: an image sensor; a microphone; and a controller coupled to the image sensor and the microphone for receiving a vocal instruction from a caregiver and for analyzing images from the image sensor in response to the vocal instruction, the controller configured to detect and identify the unknown object within the analyzed images.

According to another aspect of the present disclosure, a system is provided for detecting an object within a region of interest, the system including: an image sensor; and a controller coupled to the image sensor for receiving images, the controller receiving an instruction from a caregiver, the instruction identifying the region of interest in which the object may be located within the analyzed images, wherein the controller is configured to monitor the region of interest according to a monitoring protocol by analyzing images from the image sensor in order to detect the object within the region of interest.

According to another aspect of the present disclosure, a method is provided for detecting and monitoring an unknown object within a region of interest, the method including: acquiring images from an image sensor; using a microphone to receive a vocal instruction from a caregiver, the vocal instruction identifying the region of interest in which the unknown object is located; using a controller to analyze images from the image sensor in response to the vocal instruction; detecting and identifying the unknown object within the region of interest within the analyzed images; and monitoring the object according to a monitoring protocol.

According to another aspect of the present disclosure, a system is provided for monitoring a region of interest, the system including: an image sensor capturing images including the region of interest; and a controller coupled to the image sensor for receiving the images, the controller is configured to: analyze the images to detect and read a marker, the marker including information; look up a pre-stored monitoring protocol associated with the information; and monitor the region of interest according to the monitoring protocol by analyzing images from the image sensor.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is an electrical circuit diagram in block form of a system according to the present disclosure;

FIG. 2 is a flow chart illustrating the operation of the system shown in FIG. 1;

FIG. 3 is a pictorial representation of a patient and the system of FIG. 1; and

FIG. 4 is a close up of an exemplary region of interest of the patient shown in FIG. 3.

### DETAILED DESCRIPTION

The present illustrated embodiments reside primarily in combinations of method steps and apparatus components related to a system for detecting unknown objects proximate a patient. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

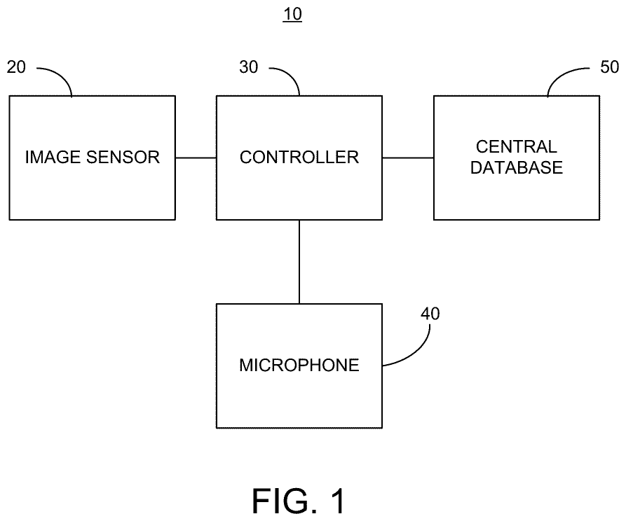

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Systems are known in the art in which a camera is placed in a patient's room of a healthcare facility for monitoring the patient and detecting objects within the room. Some systems are capable of learning the identity of unknown objects detected in the images. However, in many of these systems, the detection capability is limited to a predefined set of objects and labels. When object detection is part of a system applied to a specific environment/domain such as healthcare facility, the objects that are important to a specific case can differ by the type of healthcare unit, individual patient condition, and medical device suppliers.

The system described herein provides a real-time visual training component that can be plugged into any image-based monitoring system. At the prompt of a user or predefined criteria, the system can detect a previously unknown object and learn the object on the spot and have that new piece of information available for the rest of the system to use. In addition, the system allows for natural language processing of vocal instructions from a caregiver making the system much more intuitive to use.

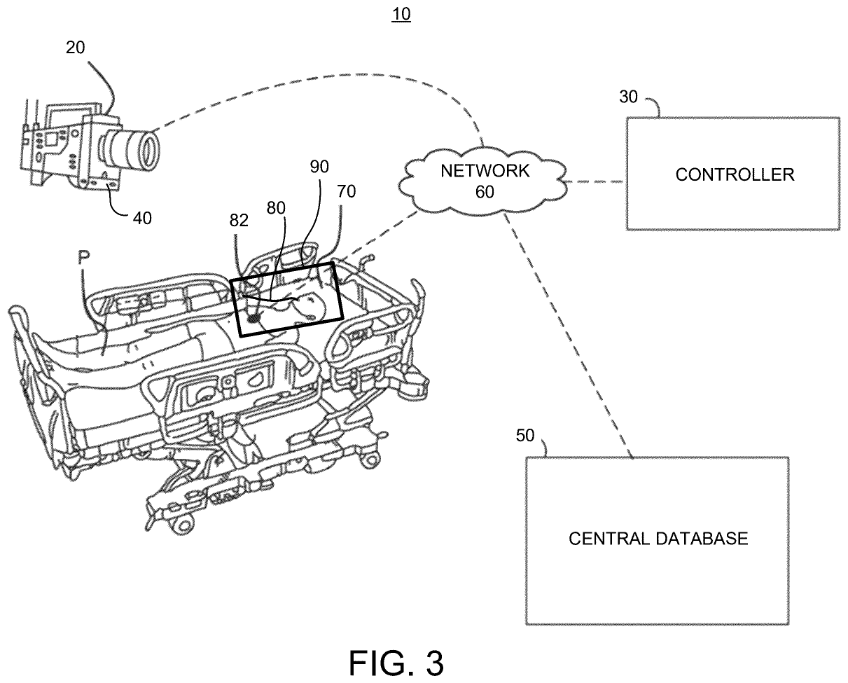

Referring to FIGS. 1 and 3, reference numeral 10 generally designates a system for detecting and/or identifying unknown objects in a region of interest. The system 10 may include an image sensor 20 and a controller 30 for analyzing images captured by the image sensor 20. According to some embodiments, the system 10 may optionally include a microphone 40 for receiving vocal instructions from a caregiver. The microphone 40 provides the vocal instructions to the controller 30, which may process and parse the vocal instructions. In other embodiments, the system 10 may be configured to alternatively or additionally receive non-vocal instructions whether entered via conventional keyboard and mouse or via gestures. As explained further below, the controller 30 may be configured to respond to such instructions by adjusting how it analyzes the images from the image sensor 20 or how it responds to the detection of an unknown object within the images.

The image sensor 20 may be a regular RGB, depth, stereo, infrared (IR), thermal image sensor, etc., or a combination thereof. Each of those options could contribute to different environments and/or accurate segmentation.

The system 10 may also be in communication with a central database 50, which may include a central image repository. This allows access of a library of images of objects with their labels such that the system 10 may more readily identify unknown objects and later make the identification of an unknown object available to other systems. The system 10 may communicate either directly or indirectly with the database 50 via a network 60 (FIG. 3).

The controller 30 may include several processing and control circuits. For example, the controller 30 may include an image processor and a voice recognition processor.

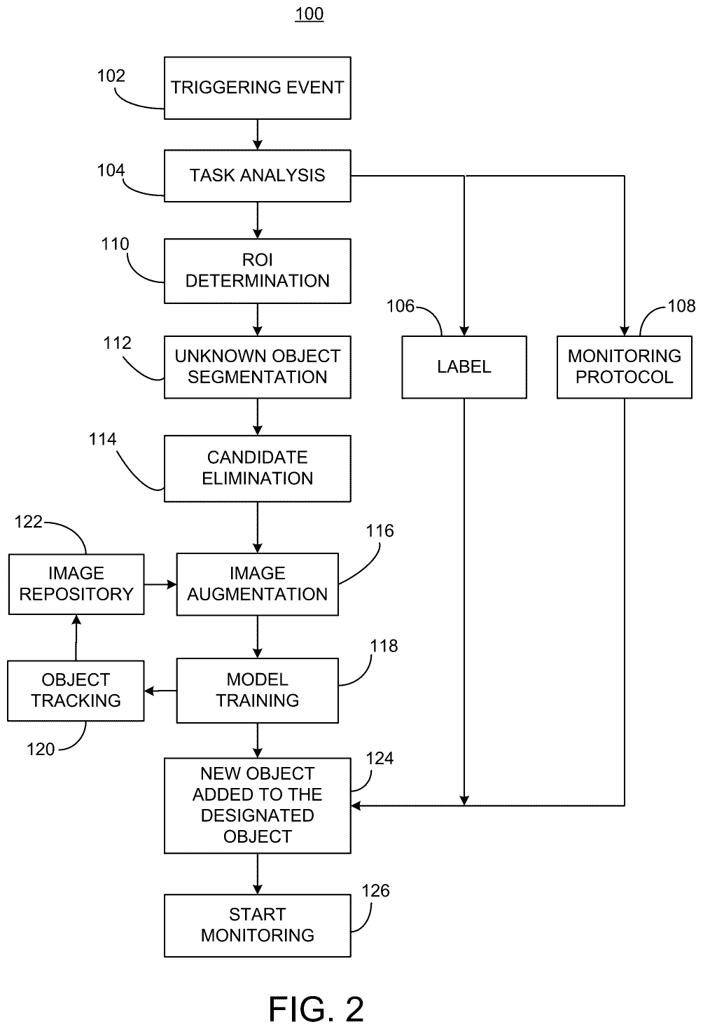

The controller 30 may be configured to execute the method 100 shown in FIG. 2. The method 100 starts with the detection of a triggering event (step 102). Such a triggering event may include any of the following examples:
1. A nurse (caregiver) says “make sure the oxygen tube stays on patient's nose.” The controller 30 detects the “unknown item” on patient's nose, registers the item around patient's nose as an oxygen tube and alerts the nurse if the item is no longer detected on that location.
2. A nurse holds out an item and says “This is a cigarette. I want this to be added to the prohibited items.”
3. An unknown object appears within a predefined region of interest (ROI) such as patient's face or other body parts.
4. An unknown object appears within the room that exceeds either size or time threshold.
5. A marker is detected and identified in the images. Such markers are described further below.

Thus, per examples 1 and 2, the triggering event may be a vocal instruction from the caregiver. The triggering event may also be detection of an object and/or a marker as a result of monitoring the images captured by the image sensor 20 per examples 3-5.

Markers, such as machine-readable text or codes (bar codes, QR codes, etc.), may be disposed on or proximate objects in order to identify the objects. The controller 30 may thus read these codes in the images captured by the image sensor 20 and identify the associated objects.

Next, in step 104, the controller 30 performs task analysis. Task analysis determines a ROI, a label, and a monitoring protocol of the unknown object. The analysis can be done via a natural language model if triggering events are vocal instructions such as in examples 1 and 2. If the event was triggered without user prompt, such as in examples 3 and 4, the system 10 will prompt the user to define a monitoring protocol and a user-facing label for the object. However, ROI will be predefined within the triggering event definition in this case. If a marker is detected and read per example 5, the monitoring protocol may be automatically determined based on the identification of the object associated with the marker.

In step 106, the controller 30 determines from the vocal instruction, the label to apply to the unknown object. In example 1, the label is “oxygen tube.” In example 2, the label is “cigarette.” In examples 3 and 4, the label is not provided because the object is unknown.

In step 108, the controller 30 determines the monitoring protocol to employ. This may come from the instruction or may be predefined for a particular label or context in the instruction. In example 1, the controller 30 determines from the vocal instruction, the monitoring protocol is to alert the nurse if the item is no longer detected on the patient's nose. In example 2, the controller 30 is not explicitly given the monitoring protocol in the instruction, but rather the controller 30 knows from the instruction that the object is “prohibited” that the protocol is to alert the nurse if the object is detected in the ROI (e.g., the room). In example 3, the monitoring protocol is already defined such as a prohibited item so that an alert is provided to the caregiver if such an unknown object is detected. The same goes for example 4. Examples of objects in examples 3 and 4 that may trigger an event are personal belongings such as jewelry, wearables, dentures, etc. or tele-packs, personal walkers, or other assistive devices. These triggering events can mainly be utilized as “object last seen” where a patient loses his/her belongings in the hospital. These personal objects are most likely not recognized as part of common objects in the room, so they are candidates for the real-time learning and tracking using the system 10. For these items, the follow-up action from the system 10 can be asking the users if they want to start tracking them, or add them as a variation to known objects, etc. As noted above, if a marker is detected and read per example 5, the monitoring protocol may be automatically determined based on the identification of the object associated with the marker. An example would be a marker on an oxygen tube that identifies the oxygen tube so that the controller 30 may then automatically select and execute an appropriate monitoring protocol for the oxygen tube such as alerting a nurse if the oxygen tube is no longer detected on the patient's nose.

In a broader sense, a system may be provided for monitoring a region of interest, the system including: an image sensor capturing images including the region of interest; and a controller coupled to the image sensor for receiving the images, the controller is configured to: analyze the images to detect and read a marker, the marker including information; look up a pre-stored monitoring protocol associated with the information; and monitor the region of interest according to the monitoring protocol by analyzing images from the image sensor. The information may include the identity of an object associated with the marker. The region of interest may be a region within the analyzed images that is less than the field of view of the image sensor. The region of interest may be a body part of one of a patient and a caregiver.

It should be noted that the marker may also be used to “register” the object, and once the object is learned, the marker may no longer be needed. Thus, the marker not only may trigger the monitoring protocol associated with the object but also may trigger the learning of the object.

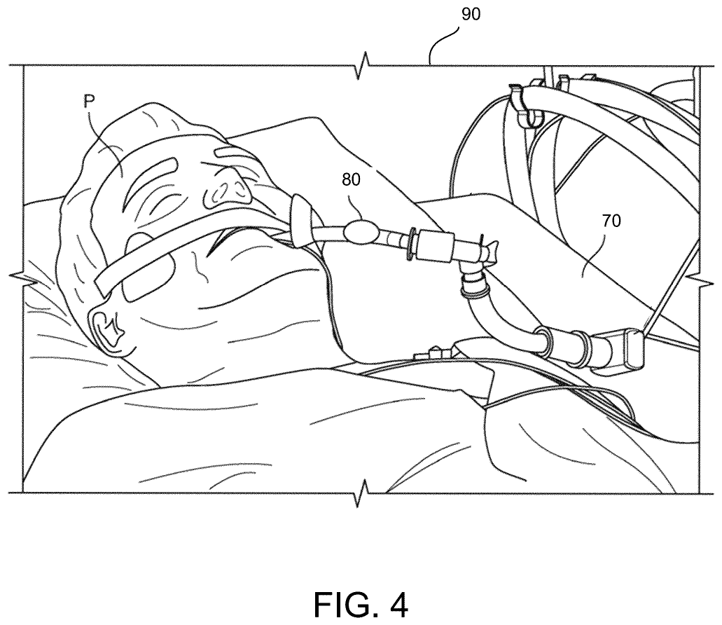

The determination of ROI (step 110) may thus be made by parsing a vocal instruction or it may simply be the room corresponding to the field of view of the image sensor 20. In example 1 above, the caregiver says, “make sure the oxygen tube stays on patient's nose.” The controller 30 determines from this instruction that the ROI is the region of the patient's nose. In example 2 above, where the caregiver says, “This is a cigarette. I want this to be added to the prohibited items,” the controller 30 determines that the region of interest is the caregiver's hand holding the object. In example 3, the ROI is predefined as the patient's nose or other body part. In example 4, the ROI is predefined as the room. In the example shown in FIGS. 3 and 4 (broadly applicable to any of the embodiments herein), the ROI 90 is the face or chest of the patient P who is lying on bed 70. In this case, the object is a tracheal intubation tube 80 or a respiratory monitor 82. The monitoring protocol may be to alert a caregiver (through, for example, the controller 30, the central database 50 and/or the network 60 or otherwise) if the tube 80 is removed from the patient's mouth or the respiratory monitor 82 is removed from the patient's chest.

Once the ROI is determined, the controller 30 may then perform unknown object segmentation in step 112. Such unknown object segmentation can be performed via a stereo imaging device or a depth camera, and/or using an unknown object segmentation model.

In the next step 114, the controller 30 may perform candidate elimination when multiple unknown objects are detected within ROI. In particular, in the event the instruction states to “make sure the oxygen tube stays on patient's nose,” and there are two unknown objects in the region around the patient's nose, it may be possible to eliminate one of the unknown objects as not being an oxygen tube by identifying the other unknown object based on the image repository or simply determining that the other object simply cannot be considered a tube of any sort.

Thereafter, image augmentation (step 116) and model training (step 118) may be performed to identify the unknown object. Model training is done using input images generated by the image augmentation. Input to the image augmentation includes:
segmented image from the current image frame;
segmented image from the future image frames-edge tracking (step 120) can be used;
image repository (from database 50) that contains background images from the current environment and images of other known objects (step 122); and
any three-dimensional (3D) models (for example, CAD or .stp files) associated with known objects.

The output of the image augmentation is used as training data. The training data then utilizes a transfer learning mechanism to train a new layer in a deep learning model-like structure where only the final few layers are learned to recognize the new object.

Once the object has been identified within the images, the new object is added to the designated object along with the label (if any) and the monitoring protocol in step 124. Data relating to the object from a plurality of views may be stored in the central database 50. The controller 30 may then start monitoring the object in accordance with the monitoring protocol in step 126.

Unlike prior systems, in the system 10, the controller 30 may analyze images from the image sensor 20 in response to a vocal instruction. The controller 30 may then detect the unknown object within the analyzed images and apply a label provided in the instruction. Further, the system 10 may receive an instruction to monitor an ROI within the analyzed images. The ROI may be a region within the analyzed images that is less than the field of view of the image sensor 20. More specifically, the ROI may be a region of one of a patient and the caregiver. Also, the monitoring protocol may be provided via the vocal instruction.

Moreover, the system 10 is designed to provide a real-time visual training component that can be plugged into any image-based monitoring system. At the prompt of a user or predefined criteria, the system can detect previously unknown objects and can learn that on the spot and have that new piece of information for the rest of the system to use.

According to a first aspect of the present disclosure, a system is provided for detecting an unknown object in a region of interest, the system including: an image sensor; a microphone; and a controller coupled to the image sensor and the microphone for receiving a vocal instruction from a caregiver and for analyzing images from the image sensor in response to the vocal instruction, the controller configured to detect and identify the unknown object within the analyzed images.

Another feature of the first aspect is that the controller monitors the object according to a monitoring protocol.

Another feature of the first aspect is that the monitoring protocol is provided in the vocal instruction.

Another feature of the first aspect is that, upon identifying the previously unknown object, the controller is configured to retrieve the monitoring protocol from a central database based on the identity of the previously unknown object.

Another feature of the first aspect is that the identity of object is provided in the vocal instruction and the controller identifies the unknown object based on the identity provided in the vocal instruction.

Another feature of the first aspect is that the controller saves data relating to the object from a plurality of views and stores the data in a central database.

Another feature of the first aspect is that the controller uses natural language voice recognition to deconstruct the voice instruction.

Another feature of the first aspect is that the vocal instruction identifies the region of interest in which the unknown object is located, and wherein the controller is configured to detect and identify the unknown object within the region of interest within the analyzed images.

Another feature of the first aspect is that the region of interest is a region within the analyzed images that is less than the field of view of the image sensor.

Another feature of the first aspect is that the region of interest is a region of one of a patient and the caregiver.

According to a second aspect of the present disclosure, a system is provided for detecting an object within a region of interest, the system including: an image sensor; and a controller coupled to the image sensor for receiving images, the controller receiving an instruction from a caregiver, the instruction identifying the region of interest in which the object may be located within the analyzed images, wherein the controller is configured to monitor the region of interest according to a monitoring protocol by analyzing images from the image sensor in order to detect the object within the region of interest.

Another feature of the second aspect is that the region of interest is a region within the analyzed images that is less than the field of view of the image sensor.

Another feature of the second aspect is that the region of interest is a body part of one of a patient and the caregiver.

Another feature of the second aspect is that the system further includes a microphone coupled to the controller, wherein the instruction is a vocal instruction received from the microphone.

Another feature of the second aspect is that the monitoring protocol is provided in the vocal instruction.

Another feature of the second aspect is that the identity of object is provided in the vocal instruction and the controller identifies the object based on the identity provided in the vocal instruction.

Another feature of the second aspect is that the controller uses natural language voice recognition to deconstruct the voice instruction.

Another feature of the second aspect is that, upon determining that an unknown object is located in the region of interest, the controller is configured to prompt the caregiver to define a monitoring protocol and a label for the unknown object.

Another feature of the second aspect is that, upon determining that an unknown object appears in a same room as the system and exceeds one or more of a size or time threshold, the controller is configured to prompt the caregiver to define a monitoring protocol and a label for the unknown object.

According to a third aspect of the present disclosure, a method is provided for detecting and monitoring an unknown object within a region of interest. The method including: acquiring images from an image sensor; using a microphone to receive a vocal instruction from a caregiver, the vocal instruction identifying the region of interest in which the unknown object is located; using a controller to analyze images from the image sensor in response to the vocal instruction; detecting and identifying the unknown object within the region of interest within the analyzed images; and monitoring the object according to a monitoring protocol.

According to a fourth aspect of the present disclosure, a system is provided for detecting an unknown object in a region of interest, the system including: an image sensor; a microphone; and a control means coupled to the image sensor and the microphone for receiving a vocal instruction from a caregiver and for analyzing images from the image sensor in response to the vocal instruction, the control means configured to detect and identify the unknown object within the analyzed images.

According to the fourth aspect, the control means comprises one or more processors programmed to perform analysis of images in response to a vocal instruction.

According to a fifth aspect of the present disclosure, a system is provided for monitoring a region of interest, the system including: an image sensor capturing images including the region of interest; and a controller coupled to the image sensor for receiving the images, the controller is configured to: analyze the images to detect and read a marker, the marker including information; look up a pre-stored monitoring protocol associated with the information; and monitor the region of interest according to the monitoring protocol by analyzing images from the image sensor.

According to the fifth aspect, the information includes the identity of an object associated with the marker.

According to the fifth aspect, the region of interest is a region within the analyzed images that is less than the field of view of the image sensor.

According to the fifth aspect, the region of interest is a body part of one of a patient and a caregiver.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A system for detecting an unknown object in a region of interest, the system comprising:
an image sensor;
a microphone; and
a controller coupled to the image sensor and the microphone for receiving a vocal instruction from a caregiver and for analyzing images from the image sensor in response to the vocal instruction, the controller configured to detect and identify the unknown object within the analyzed images.

2. The system of claim 1, wherein the controller monitors the object according to a monitoring protocol.

3. The system of claim 2, wherein the monitoring protocol is provided in the vocal instruction.

4. The system of claim 2, wherein, upon identifying the previously unknown object, the controller is configured to retrieve the monitoring protocol from a central database based on the identity of the previously unknown object.

5. The system of claim 1, wherein the identity of the object is provided in the vocal instruction and the controller identifies the unknown object based on the identity provided in the vocal instruction.

6. The system of claim 1, wherein the controller saves data relating to the object from a plurality of views and stores the data in a central database.

7. The system of claim 1, wherein the controller uses natural language voice recognition to deconstruct the voice instruction.

8. The system of claim 1, wherein the vocal instruction identifies the region of interest in which the unknown object is located, and wherein the controller is configured to detect and identify the unknown object within the region of interest within the analyzed images.

9. The system of claim 8, wherein the region of interest is a region within the analyzed images that is less than the field of view of the image sensor.

10. The system of claim 8, wherein the region of interest is a region of one of a patient and the caregiver.

11. A system for detecting an object within a region of interest, the system comprising:
an image sensor; and
a controller coupled to the image sensor for receiving images, the controller receiving an instruction from a caregiver, the instruction identifying the region of interest in which the object may be located within the analyzed images, wherein the controller is configured to monitor the region of interest according to a monitoring protocol by analyzing images from the image sensor in order to detect the object within the region of interest.

12. The system of claim 11, wherein the region of interest is a region within the analyzed images that is less than the field of view of the image sensor.

13. The system of claim 11, wherein the region of interest is a body part of one of a patient and the caregiver.

14. The system of claim 11, and further comprising a microphone coupled to the controller, wherein the instruction is a vocal instruction received from the microphone.

15. The system of claim 14, wherein the monitoring protocol is provided in the vocal instruction.

16. The system of claim 14, wherein the identity of the object is provided in the vocal instruction and the controller identifies the object based on the identity provided in the vocal instruction.

17. The system of claim 14, wherein the controller uses natural language voice recognition to deconstruct the voice instruction.

18. The system of claim 11, wherein, upon determining that an unknown object is located in the region of interest, the controller is configured to prompt the caregiver to define a monitoring protocol and a label for the unknown object.

19. The system of claim 11, wherein, upon determining that an unknown object appears in the same room as the system and exceeds one or more of a size or time threshold, the controller is configured to prompt the caregiver to define a monitoring protocol and a label for the unknown object.

20. A method of detecting and monitoring an unknown object within a region of interest, the method comprising:
acquiring images from an image sensor;
using a microphone to receive a vocal instruction from a caregiver, the vocal instruction identifying the region of interest in which the unknown object is located;
using a controller to analyze images from the image sensor in response to the vocal instruction;
detecting and identifying the unknown object within the region of interest within the analyzed images; and
monitoring the object according to a monitoring protocol.

21. A system for monitoring a region of interest, the system comprising:
an image sensor capturing images including the region of interest; and
a controller coupled to the image sensor for receiving the images, the controller is configured to:
analyze the images to detect and read a marker, the marker including information;
look up a pre-stored monitoring protocol associated with the information; and
monitor the region of interest according to the monitoring protocol by analyzing images from the image sensor.

22. The system of claim 21, wherein the information includes the identity of an object associated with the marker.

23. The system of claim 21, wherein the region of interest is a region within the analyzed images that is less than the field of view of the image sensor.

24. The system of claim 21, wherein the region of interest is a body part of one of a patient and a caregiver.
